(19) United States
(12) Patent Application Publication
(10) Pub. No.: US 2020/0316239 A1
(43) Pub. Date: Oct. 8, 2020

(54) PLASMA STERILIZATION AND DRYING SYSTEM AND METHODS

(71) Applicant: 3M INNOVATIVE PROPERTIES COMPANY, St. Paul, MN (US)

(72) Inventors: Sarah J. Davis, Stillwater, MN (US); Caleb T. Nelson, Woodbury, MN (US); Jodi L. Connell, St. Paul, MN (US); Joshua D. Erickson, Champlin, MN (US); Jeffrey D. Smith, Marine on St. Croix, MN (US); Jay R. Goetz, Deephaven, MN (US); Nicholas R. Powley, St. Paul, MN (US); Matthew T. Scholz, Woodbury, MN (US)

(21) Appl. No.: 15/733,310

(22) PCT Filed: Dec. 26, 2018
(86) PCT No.: PCT/IB2018/060626

§ 371 Date: Jun. 23, 2020

Related U.S. Application Data
(60) Provisional application No. 62/612,345, filed on Dec. 30, 2017.

(51) Int. Cl. A61L 2/14; A61L 2/26; H01J 37/32

(52) U.S. Cl. CPC A61L 2/14; A61L 2/26; H01J 37/32449; H01J 37/32348; H01J 2237/327; A61L 2202/122; A61L 2202/15; A61L 2202/24; A61L 2202/11

(57) ABSTRACT

A system and methods for sterilizing and drying contaminated articles, particularly medical articles, and more particularly the hollow internal areas of medical instruments or lumens of medical endoscopes. The system includes a plasma generator having an electrode, a shield, and a dielectric gap between the electrode and the shield. A source of electrical power connected to the plasma generator applies an electrode energy density between the electrode and the shield. A source of a sterilizing gas precursor provides a flow of the sterilizing gas precursor through the plasma generator to generate a plasma, thereby forming a sterilizing gas including acidic and/or oxidizing species. The contaminated article is exposed to the sterilizing gas for a time sufficient to achieve a desired degree of sterilization. A turbulent flow of a drying gas is used to dry the contaminated article alternately with the exposure of the contaminated article to the sterilizing gas.

### FIELD

The present disclosure relates generally to the sterilization or disinfection, and drying of medical apparatus and articles, and more particularly to the alternate application of a gas plasma to effect sterilization or disinfection, and a turbulent gas flow to effect drying, of medical articles such as medical instruments or medical endoscope lumens.

### BACKGROUND

A reliable supply of sterile apparatus, instruments and supplies is vitally important to modern medical practice. Various types of apparatus are known for sterilizing reusable goods within a hospital setting including, for example, steam autoclaves. U.S. Pat. No. 4,301,113 (Alguire et al); U.S. Pat. No. 4,294,804 (Baran); U.S. Pat. No. 5,317,896 (Sheth et al); U.S. Pat. No. 5,399,314 (Samuel et al); U.S. Pat. No. 3,571,563 (Shulz); U.S. Pat. No. 3,054,270 (Huston); and U.S. Pat. No. 3,564,861 (Andersen et al), discuss sterilization apparatus and their control systems. Goods which cannot withstand autoclaving temperatures can be sterilized with sterilizers using a biocidal gas such as ethylene oxide.

Although ethylene oxide is an excellent sterilant and penetrates well into the lumens of, e.g., endoscopes, ethylene oxide also exhibits undesirable toxicity and flammability, and for at least these reasons, the art has sought alternatives.

### SUMMARY

The present disclosure provides a sterilization or disinfection and drying system employing an oxygen/nitrogen plasma to effect sterilization or disinfection and a turbulent gas flow to effect drying of medical articles such as medical instruments or medical endoscope lumens. The disclosed embodiments permit a high electrode energy density while minimizing unwanted heat production. The disclosed embodiments achieve removal of all visible moisture from the lumen channels of medical endoscopes in addition to demonstrating effective sterilization by obtaining full kill (6-7 log 10) of a representative model organism relevant to endoscope reprocessing.

Thus, in one aspect, the present disclosure relates to a system for sterilizing a contaminated article including a source of a drying gas configured to provide a turbulent flow of the drying gas to dry the contaminated article; a plasma generator having an electrode, a shield, and a dielectric gap between the electrode and the shield; a source of electrical power connected to the plasma generator for applying an electrode energy density between the electrode and the shield; and a source of a sterilizing gas precursor comprising water vapor, oxygen, and nitrogen, configured to provide a flow of the sterilizing gas precursor through the plasma generator between the electrode and the shield to form a plasma. A temperature at the surface of the shield is maintained at less than 150° C. when the electrode energy density is greater than 0.05 eV/molecule of the sterilizing gas precursor passing between the electrode and the shield. The plasma forms from the sterilizing gas precursor a sterilizing gas comprising acidic and/or oxidizing species. The contaminated article is exposed to a flow of the sterilizing gas.

In exemplary embodiments of the system, the sterilizing gas includes one or more species selected from the group consisting of molecular oxygen, molecular nitrogen, nitric oxide, nitric acid, and nitrous oxide. Preferably, the sterilizing gas precursor includes water vapor, molecular oxygen, and molecular nitrogen. In some exemplary embodiments, the sterilizing gas precursor comprises air. Preferably, the relative humidity of the sterilizing gas precursor entering the plasma generator is at least 21%.

In certain presently preferred embodiments, the temperature at the surface of the shield is maintained at less than 150° C. when the electrode energy density is greater than 0.05 eV/molecule of the gas passing between the electrode and the shield. In some exemplary embodiments, the source of electrical power is a pulsed DC source having a high dV/dT.

Optionally, the system further includes a device for conveying the contaminated article through a chamber fluently connected to the flow of the sterilizing gas. In certain exemplary embodiments, the system further includes a cooling apparatus. In some exemplary embodiments, the system includes a filter for removing the acidic and/or oxidizing species from the sterilizing gas.

In a second aspect, the present disclosure describes a method for sterilizing a contaminated article using a sterilizer, the method including providing a sterilizer including: a source of a drying gas configured to provide a turbulent flow of the drying gas to dry the contaminated article; a plasma generator including an electrode, a shield, and a dielectric gap between the electrode and the shield; a source of electrical power connected to the plasma generator for applying an electrode energy density between the electrode and the shield; and a source of a sterilizing gas precursor comprising water vapor, oxygen, and nitrogen, configured to provide a flow of the sterilizing gas precursor through the plasma generator between the electrode and the shield to form a plasma containing acidic and/or oxidizing species from the sterilizing gas precursor. The method further includes providing the flow of the sterilizing gas precursor through the plasma generator between the electrode and the shield to form the plasma, wherein a temperature at the surface of the shield is maintained at less than 150° C. when the electrode energy density is greater than 0.05 eV/molecule of the sterilizing gas precursor passing between the electrode and the shield. The plasma causes the flow of sterilizing gas precursor to form a flow of a sterilizing gas comprising the acidic and/or oxidizing species. The method further includes directing the flow of the sterilizing gas containing the acidic and/or oxidizing species from the plasma generator through an enclosed space enclosing at least a portion of the contaminated article, exposing the contaminated article to the sterilizing gas containing the acidic and/or oxidizing species for an exposure time sufficient to achieve a desired degree of sterilization of the contaminated article, and directing a turbulent flow of the drying gas into the enclosed space to dry the contaminated article.

In some particular exemplary embodiments, the contaminated article is exposed to the gas containing the acidic and/or oxidizing species for an exposure time sufficient to achieve the desired degree of sterilization of the contaminated article, which is preferably no more than one hour.

In certain presently-preferred embodiments, directing the flow of the sterilizing gas through the enclosed space occurs for a duration of at least 10 sec and no more than 5 min, and is followed by directing the flow of the drying gas through the enclosed space for a duration of at least 10 sec and no more than 10 min. Preferably, this process of alternately directing the flow of the sterilizing gas through the enclosed space and directing the flow of the drying gas through the enclosed space, is repeated at least twice.

In additional exemplary embodiments, the sterilizing gas precursor includes water vapor, oxygen, and nitrogen, and the temperature at the surface of the shield is maintained at less than 150° C. when the electrode energy density is greater than 0.05 eV/molecule of the gas passing between the electrode and the shield. In further exemplary embodiments, the drying gas is selected from the group consisting of oxygen, nitrogen, helium, neon, argon, krypton, or a combination thereof, optionally wherein the drying gas is substantially free of water. In certain exemplary embodiments, at least one of the drying gas, the sterilizing gas precursor, or the sterilizing gas has a temperature of from 10° C. to 60° C.

In some particular exemplary embodiments, the contaminated article is a medical device and the enclosed space is a hollow area of the medical device. In some such embodiments, the medical device is an endoscope and the hollow area is a lumen of the endoscope, further wherein the sterilizing gas containing the acidic and/or oxidizing species from the plasma generator is passed through the lumen of the endoscope. In other exemplary embodiments, the medical device is a medical instrument and the hollow area is at least one internal cavity of the medical instrument.

In certain exemplary embodiments, the contaminated article is contaminated with at least one of a bio-film comprised of a plurality of microorganisms, a plurality of microorganisms, a bio-film comprised of a plurality of microbial spores, a plurality of microbial spores, a bio-film comprised of a plurality of fungal spores, or a plurality of fungal spores. These organisms may be present along with biological soil such as blood, feces, mucous and the like. In some such exemplary embodiments, the bio-film comprises a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Streptococcus mutans Shigella flexiniri, and combinations thereof.

In some exemplary embodiments, the contaminated article is contaminated with a bio-film comprising a plurality of microorganisms, further wherein the exposure time is at least 5 minutes, and the reduction in colony forming units of the disinfected article relative to the contaminated article is from 4-log₁₀ to 9-log₁₀, optionally wherein the exposure time is at most one hour.

In further exemplary embodiments, the contaminated article is contaminated with a bio-film comprising a plurality of microbial or fungal spores, further wherein the exposure time is at least 2 minutes, and the reduction in colony forming units of the disinfected article relative to the contaminated article is from 6-log₁₀ to 10-log₁₀, optionally wherein the exposure time is at most one hour.

Additional exemplary embodiments within the scope of the present disclosure are provided in the following Listing of Exemplary Embodiments.

### Listing of Exemplary Embodiments

A. A system for sterilizing a contaminated article, comprising:
a source of a drying gas configured to provide a turbulent flow of the drying gas to dry the contaminated article;
a plasma generator having:
   an electrode,
   a shield, and
   a dielectric gap between the electrode and the shield;
a source of electrical power connected to the plasma generator for applying an electrode energy density between the electrode and the shield; and
a source of a sterilizing gas precursor comprising water vapor, oxygen, and nitrogen, configured to provide a flow of the sterilizing gas precursor through the plasma generator between the electrode and the shield to form a plasma, wherein a temperature at the surface of the shield is maintained at less than 150° C. when the electrode energy density is greater than 0.05 eV/molecule of the sterilizing gas precursor passing between the electrode and the shield, further wherein the plasma forms from the sterilizing gas precursor a sterilizing gas comprising acidic and/or oxidizing species, and further wherein the contaminated article is exposed to a flow of the sterilizing gas, optionally wherein the system further comprises a device for conveying the contaminated article through a chamber fluently connected to the flow of the sterilizing gas.
   B. The system of Embodiment A, wherein the sterilizing gas includes one or more species selected from the group consisting of molecular oxygen, molecular nitrogen, nitric oxide, nitric acid, and nitrous oxide.
   C. The system of Embodiment A or B, wherein the sterilizing gas precursor comprises air, optionally wherein a relative humidity of the sterilizing gas precursor is at least 21%.
   D. The system of any one of Embodiments A-C, further comprising one or more valves configured to alternate the flow of the drying gas and the flow of the sterilizing gas to the contaminated article.
   E. The system of any one of Embodiments A-D, further comprising a cooling apparatus.
   F. The system of any one of Embodiments A-E, wherein the source of electrical power is a pulsed DC source having a high dV/dT.
   G. The system of any one of Embodiments A-F, further comprising a filter for removing the acidic and/or oxidizing species from the sterilizing gas.
   H. A method of sterilizing a contaminated article, comprising:
providing a sterilizer including:
   a source of a drying gas configured to provide a turbulent flow of the drying gas to dry the contaminated article;
a plasma generator including:
   an electrode,
   a shield, and
   a dielectric gap between the electrode and the shield;
a source of electrical power connected to the plasma generator for applying an electrode energy density between the electrode and the shield; and
a source of a sterilizing gas precursor comprising water vapor, oxygen, and nitrogen, configured to provide a flow of the sterilizing gas precursor through the plasma generator between the electrode and the shield to form a plasma containing acidic and/or oxidizing species from the sterilizing gas precursor;
providing the flow of the sterilizing gas precursor through the plasma generator between the electrode and the shield to form the plasma, wherein a temperature at the surface of the shield is maintained at less than 150° C. when the electrode energy density is greater than 0.05 eV/molecule of the sterilizing gas precursor passing between the electrode and the shield, further wherein the plasma causes the flow of sterilizing gas precursor to form a flow of a sterilizing gas comprising the acidic and/or oxidizing species;
directing the flow of the sterilizing gas containing the acidic and/or oxidizing species from the plasma generator through an enclosed space enclosing at least a portion of the contaminated article;
exposing the contaminated article to the sterilizing gas containing the acidic and/or oxidizing species for an exposure time sufficient to achieve a desired degree of sterilization of the contaminated article, optionally wherein the time sufficient to achieve the desired degree of sterilization of the contaminated article is no greater than one hour; and
directing a turbulent flow of the drying gas into the enclosed space to dry the contaminated article.
   I. The method of Embodiment H, further comprising removing at least a portion of the acidic and/or oxidizing species from the sterilizing gas after the sterilizing gas is directed through the enclosed space.
   J. The method of Embodiment I, wherein the removing at least a portion of the acidic and/or oxidizing species from the sterilizing gas is performed with a filter comprising one or more materials selected from the group consisting of activated carbon, a species with a basic functionality, a species providing a basic adsorbent, a reducing species, and a molecular sieve.
   K. The method of any one of Embodiments H-J, wherein the enclosed space is a sterilization chamber into which the contaminated article is placed.
   L. The method of any one of Embodiments H-K, wherein the directing the flow of the sterilizing gas through the enclosed space occurs for a duration of at least 10 sec and no more than 5 min, and is followed by the directing the flow of the drying gas through the enclosed space for a duration of at least 10 sec and no more than 10 min, optionally wherein the directing the flow of the sterilizing gas and the directing the flow of the drying gas are alternately repeated at least twice.
   M. The method of any one of Embodiments H-L, wherein at least one of the drying gas, the sterilizing gas precursor, or the sterilizing gas has a temperature of from 10° C. to 60° C.
   N. The method of any one of claims Embodiments H-M, wherein the drying gas is selected from the group consisting of oxygen, nitrogen, helium, neon, argon, krypton, or a combination thereof, optionally wherein the drying gas is substantially free of water.
   O. The method of any one of Embodiments H-N, wherein the sterilizing gas includes one or species selected from the group consisting of molecular oxygen, molecular nitrogen, nitric oxide, nitric acid, and nitrous oxide.
   P. The method of any one of Embodiments H-O, wherein the sterilizing gas precursor comprises air, optionally wherein a relative humidity of the sterilizing gas precursor entering the plasma generator is at least 21%.
   Q. The method of any one of Embodiments H-P, wherein the source of electrical power is a pulsed DC source having a high dV/dT.
   R. The method of any one of Embodiments H-Q, wherein the contaminated article is a medical device and the enclosed space is a hollow area of the medical device.
   S. The method of Embodiment R, wherein the medical device is an endoscope and the hollow area is a lumen of the endoscope, further wherein the sterilizing gas containing the acidic and/or oxidizing species from the plasma generator is passed through the lumen of the endoscope.
   T. The method of Embodiment R, wherein the medical device is a medical instrument and the hollow area is at least one internal cavity of the medical instrument.
   U. The method of any one of Embodiments H-T, wherein the contaminated article is contaminated with at least one of a bio-film comprised of a plurality of microorganisms, a plurality of microorganisms, a bio-film comprised of a plurality of microbial spores, a plurality of microbial spores, a bio-film comprised of a plurality of fungal spores, or a plurality of fungal spores.
   V. The method of Embodiment U, wherein the bio-film comprises a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Streptococcus mutans, Shigella flexiniri, and combinations thereof.
   W. The method of Embodiment U or V, wherein the contaminated article is contaminated with a bio-film comprising a plurality of microorganisms, further wherein the exposure time is at least 5 minutes, and the reduction in colony forming units of the disinfected article relative to the contaminated article is from 4-log₁₀ to 9-log₁₀, optionally wherein the exposure time is at most one hour.
   X. The method of any one of any one of Embodiments U-W, wherein the contaminated article is contaminated with a bio-film comprising a plurality of microbial or fungal spores, further wherein the exposure time is at least 2 minutes, and the reduction in colony forming units of the disinfected article relative to the contaminated article is from 6-log₁₀ to 10-log₁₀, optionally wherein the exposure time is at most one hour.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in view of the following detailed description of exemplary embodiments with the accompanying figures, in which:

FIG. 1 is a schematic view of an exemplary sterilization and drying system of the present disclosure.

FIG. 2a is a cross-section view of one variant of a plasma generator taken along section lines 2-2 in FIG. 1.

FIG. 2b is a cross-section view of another variant of a plasma generator taken along section lines 2-2 in FIG. 1.

FIG. 2c is a cross-section view of another variant of a plasma generator taken along section lines 2-2 in FIG. 1.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

The present disclosure describes an apparatus and methods for sterilizing or disinfecting and drying articles using a gas plasma including oxygen, nitrogen, and reactive species produced from these gases. In some convenient embodiments, the plasma is directed to a chamber in which a contaminated article to be sterilized or disinfected is placed. In other convenient embodiments, the plasma is directed into a hollow area of an apparatus or article requiring sterilization or disinfection.

### Glossary

Certain terms are used throughout the description and the claims that, while for the most part are well known, may require some explanation. It should be understood that, as used herein, unless a different definition is expressly provided in the claims or elsewhere in the specification, including the drawings:

As used herein the term “sterilizing gas” refers to a gas with antimicrobial activity for treating a device or article whether or not the treated device or article is, in fact, sterilized. Sterility will depend upon many process parameters such as exposure time, initial bioburden, type of organism present, presence of soil contamination, etc. as taught herein.

As used herein the terms “disinfect” or “disinfecting” refer to a reduction in the microbial load on an article by exposure to a sterilizing gas.

The terms “about” or “approximately” with reference to a numerical value or a shape means+/−five percent of the numerical value or property or characteristic, but expressly includes the exact numerical value. For example, a viscosity of “about” 1 Pa-sec refers to a viscosity from 0.95 to 1.05 Pa-sec, but also expressly includes a viscosity of exactly 1 Pa-sec.

As used in this specification and the appended embodiments, the singular forms “a”, “an”, and “the” include plural referents unless the content clearly dictates otherwise. Thus, for example, reference to fine fibers containing “a compound” includes a mixture of two or more compounds. As used in this specification and the appended embodiments, the term “or” is generally employed in its sense including “and/or” unless the content clearly dictates otherwise.

The term “substantially” with particular reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

As used in this specification, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Exemplary Sterilizing Apparatus and Processes

The present disclosure describes a system for sterilizing a contaminated article including a source of a drying gas configured to provide a turbulent flow of the drying gas to dry the contaminated article; a plasma generator having an electrode, a shield, and a dielectric gap between the electrode and the shield; a source of electrical power connected to the plasma generator for applying an electrode energy density between the electrode and the shield; and a source of a sterilizing gas precursor comprising water vapor, oxygen, and nitrogen, configured to provide a flow of the sterilizing gas precursor through the plasma generator between the electrode and the shield to form a plasma. A temperature at the surface of the shield is maintained at less than 150° C. when the electrode energy density is greater than 0.05 eV/molecule of the sterilizing gas precursor passing between the electrode and the shield. The plasma forms from the sterilizing gas precursor a sterilizing gas comprising acidic and/or oxidizing species. The contaminated article is exposed to a flow of the sterilizing gas. In some embodiments, the system includes a device, such as a conveyor belt, for conveying the contaminated article through a chamber fluently connected to the flow of the sterilizing gas.

The present disclosure also describes a method for sterilizing a contaminated article using a sterilizer, the method including providing a sterilizer including: a source of a drying gas configured to provide a turbulent flow of the drying gas to dry the contaminated article; a plasma generator including an electrode, a shield, and a dielectric gap between the electrode and the shield; a source of electrical power connected to the plasma generator for applying an electrode energy density between the electrode and the shield; and a source of a sterilizing gas precursor comprising water vapor, oxygen, and nitrogen, configured to provide a flow of the sterilizing gas precursor through the plasma generator between the electrode and the shield to form a plasma containing acidic and/or oxidizing species from the sterilizing gas precursor.

The method further includes providing the flow of the sterilizing gas precursor through the plasma generator between the electrode and the shield to form the plasma, wherein a temperature at the surface of the shield is maintained at less than 150° C. when the electrode energy density is greater than 0.05 eV/molecule of the sterilizing gas precursor passing between the electrode and the shield. The plasma causes the flow of sterilizing gas precursor to form a flow of a sterilizing gas comprising the acidic and/or oxidizing species.

The method further includes directing the flow of the sterilizing gas containing the acidic and/or oxidizing species from the plasma generator through an enclosed space enclosing at least a portion of the contaminated article, exposing the contaminated article to the sterilizing gas containing the acidic and/or oxidizing species for an exposure time sufficient to achieve a desired degree of sterilization of the contaminated article, and directing a turbulent flow of the drying gas into the enclosed space to dry the contaminated article.

In some embodiments, the contaminated article is a medical device and the enclosed space is a hollow area of the medical device. In some such embodiments, the medical device is an endoscope and the hollow area is a lumen of the endoscope, further wherein the sterilizing gas containing the acidic and/or oxidizing species from the plasma generator is passed through the lumen of the endoscope. In other exemplary embodiments, the medical device is a medical instrument and the hollow area is at least one internal cavity of the medical instrument. In other embodiments, the enclosed space is an enclosed chamber, such as a sterilization chamber into which a contaminated article to be sterilized has been placed.

Various exemplary embodiments of the disclosure will now be described with particular reference to the Drawings.

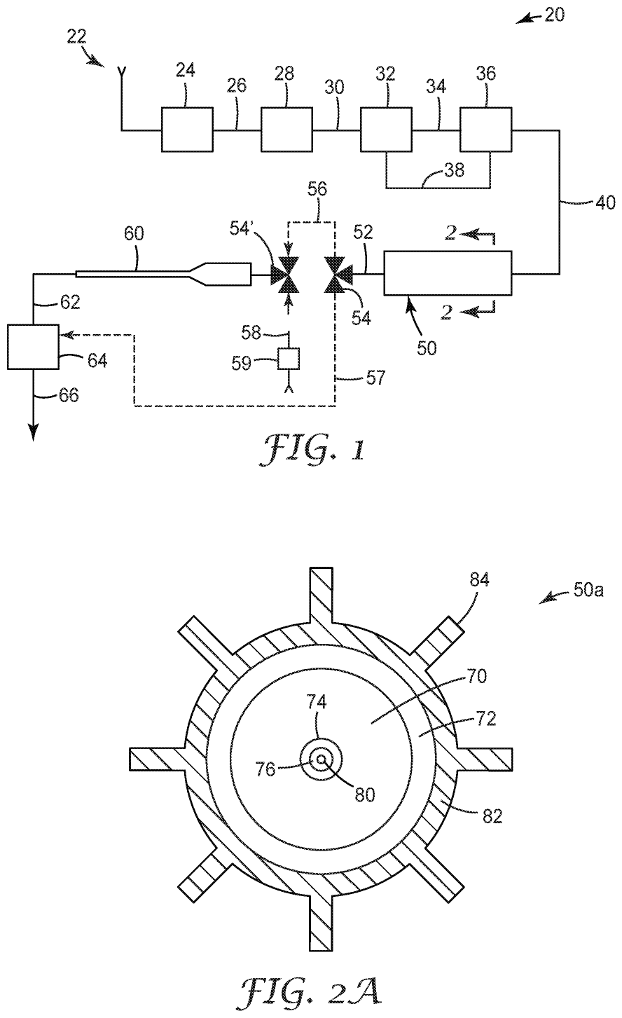

Referring now to FIG. 1, a schematic view of an exemplary sterilization or disinfection and drying system 20 of the present disclosure is illustrated. Sterilization/disinfection system 20 includes a source of sterilizing gas precursor 22, which comprises molecular oxygen and nitrogen. The sterilizing gas precursor from source 22 may be air or a specific blend including molecular oxygen and nitrogen at a specified ratio, and may be pressurized or unpressurized as provided. If from an unpressurized source 22, a compressor 24 may be used to pressurize the sterilizing gas precursor to a convenient pressure. The sterilizing gas precursor is then transported via line 26 to a flow controller 28 to meter the mass flow of sterilizing gas precursor to the rest of the sterilization system 20. Flow controller 28 may take the form of a pressure regulator, a ball-in-tube flowmeter, an electronic mass flow controller, or other similar device.

The sterilizing gas precursor is then transported via line 30 to a humidification device 32 to bring the humidity of the sterilizing gas precursor to between about 1 and 50 g/m³, between 2 and 40 g/m³, between 3 and 30 g/m³, between 4 and 20 g/m³, or even between 5 and 15 g/m³. Diverse expedients such a bubblers, spargers, atomizers, ultrasonic and wick-type humidifiers are all suitable. In the depicted embodiment, the humidified sterilizing gas precursor is conveyed via line 34 to an optional humidity detector 36 to verify that the humidity level is within the desired range. In some convenient embodiments, feedback control via control line 38 is provided to manipulate humidification device 30 appropriately.

The humidified sterilizing gas precursor is transported via line 40 to a plasma generator 50, which will be discussed with more particularity below. Plasma generator 50 induces the production of a sterilizing gas including diverse chemical species from the humidified sterilizing gas precursor, including one or more of nitrous acid, nitric acid, ozone, and nitrous oxide. This sterilizing gas is conveyed to a remote location by line 52. Surprisingly, line 52 may be quite long without losing sterilizing efficacy; distances between about 0.5 to 90 meters have been found to be suitable.

Line 52 may, for example, deliver sterilizing gas directly to an endoscope 60 to sterilize the internal lumen, or to another enclosed chamber such as a sterilization chamber (not shown in FIG. 1) into which a contaminated article to be sterilized is placed.

A source of drying gas is connected to a flow controller 59 which is connected by line 58 to the endoscope 60 or to another enclosed chamber such as a sterilization chamber (not shown in FIG. 1) into which a contaminated article to be sterilized is placed. The flow controller 59 may be any device for regulating the flowrate of the drying gas 26. Suitable devices include pressure regulators, flow control valves, ball-in-tube flowmeters (rotameters), electronic mass flow controllers, or other similar devices. The flow controller 59 is used to adjust the flowrate of the drying gas to ensure that the gas is in turbulent flow when it passes through the endoscope 60 or through another enclosed chamber such as a sterilization chamber (not shown in FIG. 1) into which a contaminated article undergoing sterilization has been placed.

Turbulent flow may be achieved when the flowrate of the drying gas through line 58 is such that the characteristic Reynolds number is greater than about 2100. The Reynolds number is defined as:

Re=(2Qp/μπR)
wherein: Q is the volumetric flowrate of the drying gas;
   ρ is the density of the drying gas;
   μ is the viscosity of the drying gas;
   and R is the radius of line 58, which has a circular cross-section

Flows of the sterilizing gas and the drying gas are alternately provided to the endoscope 60 or to another enclosed chamber such as a sterilization chamber (not shown in FIG. 1) into which a contaminated article to be sterilized is placed. Alternating the flow of the sterilizing gas with the flow of the drying gas may be advantageously carried out using three-way valves 54 and 54′, which advantageously may be electronically-controlled valves such as three-way solenoid valves. In the first position of three-way valves 54 and 54′, the flow of sterilizing gas is directed from line 52 through line 56 and into the endoscope 60 or to another enclosed chamber such as a sterilization chamber (not shown in FIG. 1); and the flow of the drying gas is isolated from the endoscope 60 another enclosed chamber. After passing through the endoscope 60 or through another enclosed chamber, sterilizing gas leaves the endoscope 60 (or equivalently the enclosed chamber), via line 62 and is conveyed to a filter 64 to render the sterilizing gas harmless.

In the second position of three-way valves 54 and 54′, the turbulent flow of drying gas passes through line 58 and into endoscope 60 or another enclosed chamber, and the sterilizing gas is directed from line 52 through line 57 and into a filter 64. In convenient embodiments, the filter 64 will include an alkaline material such as sodium bicarbonate, potassium carbonate, sodium phosphate and the like, to neutralize any remaining acidic species. Preferably the alkaline material is one which when mixed with water at a concentration of 10% wt/wt in deionized water, has a pH at 23° C. of greater than 8. An element such as activated carbon to remove oxidizing species such as ozone is also conveniently present. After filtration, the sterilizing gas can be released to ambient conditions via outlet 66.

In some embodiments, directing the flow of the sterilizing gas through the enclosed space occurs for a duration of at least 10 sec (15 sec, 20 sec, 25 sec, 30 sec; 1 min, 2 min, 5 min) and no more than 5 min (4 min, 3 min, 2.5 min, 2 min), and is followed by the directing the flow of the drying gas through the enclosed space for a duration of at least 10 sec (15 sec, 20 sec, 25 sec, 30 sec; 1 min, 2 min, 5 min) and no more than 10 min (9 min, 8 min, 7 min, 6 min, 5 min, 4 min, 3 min). Preferably, alternating the flow of the sterilizing gas and the flow of the drying gas through the endoscope 60 or through another enclosed chamber is repeated at least twice (three times, four times, five times, six times, or more).

In some embodiments, at least one of the drying gas, the sterilizing gas precursor, or the sterilizing gas has a temperature of from 10° C. to 60° C.

The drying gas may be selected from oxygen, nitrogen, helium, neon, argon, krypton, or a combination thereof. Preferably, the drying gas is substantially or even entirely free of water.

The sterilizing gas precursor comprises water vapor, oxygen, and nitrogen. In some embodiments, the sterilizing gas precursor comprises air. Preferably the relative humidity of the sterilizing gas precursor entering the plasma generator is at least 21%, 22%, 23%, 24%, 25% or even higher.

The sterilizing gas includes one or species selected from the group consisting of molecular oxygen, molecular nitrogen, nitric oxide, nitric acid, and nitrous oxide.

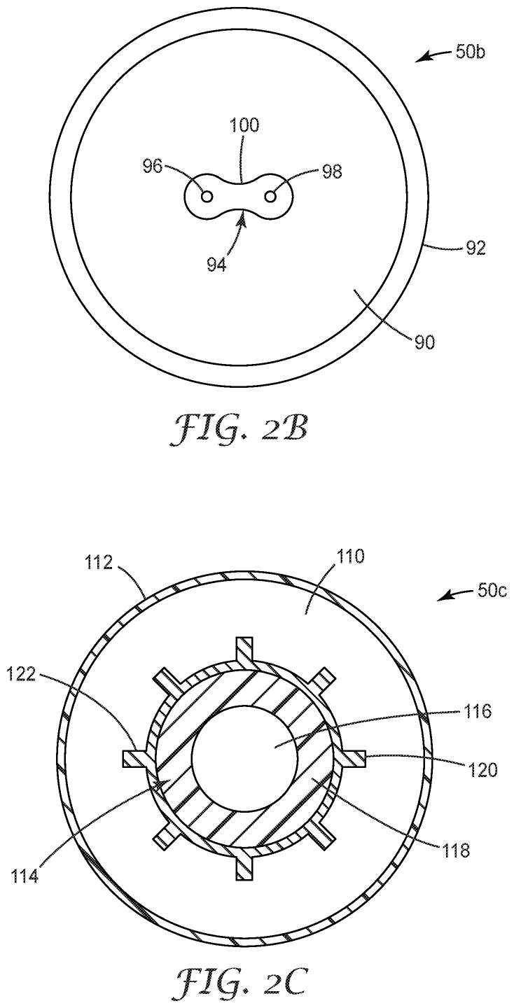

Referring now to FIG. 2a, a cross-section view of one variant 50a of plasma generator 50 taken along section lines 2-2 in FIG. 1 is illustrated. In variant 50a, the sterilizing gas is conveyed through lumen 70 in outer tube 72. Tube 72 is a dielectric, conveniently glass. Within lumen 70 is inner tube 74 having a lumen 76. Tube 74 is also a dielectric, conveniently glass. Within lumen 76 is first electrode 80. A second electrode 82 surrounds the outer tube 72, and in some convenient embodiments has heat radiating fins 84 so that it serves additional duty as a heat sink. Other expedients may be used to provide cooling, such as a fan, fins, heat exchanger, piezoelectric cooling, and combinations thereof.

During operation, a potential difference must exist between first electrode 80 and second electrode 82. In some convenient embodiments, first electrode 80 is the high voltage electrode and second electrode 82 is the ground electrode. An AC voltage of between about 4 to 12 kV is conveniently applied to first electrode 80, having a frequency of between about 4 to 30 kHz. The exact conditions depend on the gas flow needed to efficaciously treat the apparatus needing sterilization, the available cooling capacity for plasma generator 50, and the dimension of the outer and inner tubes 72 and 74 respectively. In any case, the electrical parameters must cause the conditions to exceed the breakdown voltage of the sterilizing gas precursor between the tubes.

Referring now to FIG. 2b, a cross-section view of another variant 50b of plasma generator 50 taken along section lines 2-2 in FIG. 1 is illustrated. In variant 50b, the sterilizing gas is conveyed through lumen 90 in tube 92. Tube 92 is conveniently polymeric tubing such a polytetrafluoroethylene (PTFE). Also within lumen 90 is, e.g. a ribbon cable 94 including a first conductor 96, a second conductor 98, conveniently both within a dielectric insulation 100.

Referring now to FIG. 2c, a cross-section view of one variant 50c of plasma generator 50 taken along section lines 2-2 in FIG. 1 is illustrated. In variant 50c, the sterilizing gas is conveyed through lumen 110 in tube 112. Tube 112 is conveniently polymeric tubing such a polytetrafluoroethylene (PTFE). Also within lumen 110 is electrode subassembly 114, comprising electrode 116, conveniently the high voltage electrode, surrounded by a dielectric layer 118. Around dielectric layer 118 is another electrode 120, conveniently the ground electrode. Fins 122 may conveniently be present to improve the electric field being generated.

During operation, a potential difference should exist first conductor 96 and second conductor 98. In some convenient embodiments, first conductor 96 is the high voltage electrode and second conductor 98 is the ground electrode. A DC voltage of at least 20 kV, at least 30 kV, at least 40 kV, and even at least 50 kV, but preferably no more than 100 kV, 90 kV, 80 kV, 70 kV, or even 60 kV, is conveniently applied to first conductor 96 in pulses having a duration on the order of nanoseconds with an extremely fast (i.e., high) dV/dt. By this it is meant that the rise of the pulse the highest instantaneous rate of change of the voltage should reach a rate of at least 10 kV/nano-sec, at least 20 kV/nano-sec, at least 30 kV/nano-sec, at least 40 kV/nano-sec, or even at least 50 kV/nano-sec. This type of charging allows plasma to be generated within the sterilizing gas precursor with relatively little heating.

Other exemplary embodiments of the present disclosure describe a method for sterilizing a contaminated article using a sterilization system as previously described. The sterilization system includes a plasma generator having an electrode, a shield, and a dielectric gap between the electrode and the shield, a source of electrical power connected to the plasma generator for applying an electrode energy density between the electrode and the shield, and a source of sterilizing gas precursor providing a flow through the plasma generator to form a plasma and produce a sterilizing gas containing acidic and/or oxidizing species from the sterilizing gas precursor. The sterilizing gas containing the acidic and/or oxidizing species is directed from the plasma generator into an enclosed area including the portions of the article undergoing sterilization.

In certain presently-preferred embodiments, the sterilizing gas precursor includes water vapor, oxygen, and nitrogen, and the temperature at the surface of the shield is maintained at less than 150° C. when the electrode energy density is greater than 0.05 eV/molecule of the sterilizing gas precursor passing between the electrode and the shield. The contaminated article is exposed to the sterilizing gas containing the acidic and/or oxidizing species for an exposure time sufficient to sterilize the contaminated article, which is preferably no more than one hour.

In certain exemplary embodiments, the method further includes removing at least a portion of the acidic and/or oxidizing species from the sterilizing gas upon achieving the desired degree of sterilization of the article. Removing the acidic and/or oxidizing species from the sterilizing gas may be performed with an apparatus including one or more adsorbent or absorbent materials selected from activated carbon, a chemical species with a basic functionality (e.g., an organic amine, sodium hydroxide, potassium hydroxide, lithium hydroxide, and the like), a species providing a basic adsorbent (e.g. a basic ion exchange resin), a reducing species (e.g., one or more active metals such as platinum, palladium, and the like), and a molecular sieve. In some exemplary embodiments, removing the acidic and/or oxidizing species from the sterilizing gas may be performed by directing the sterilizing gas through a catalytic reducer.

In further exemplary embodiments, the enclosed area is a sterilization chamber. In other exemplary embodiments, the article undergoing sterilization is a medical device and the enclosed area is a hollow area of the medical device. In some presently-preferred embodiments, the medical device is an endoscope and the hollow area is the lumen of the endoscope, and the sterilizing gas containing the acidic and/or oxidizing species from the plasma generator is passed through the lumen of the endoscope.

In other exemplary embodiments, the medical device is a medical instrument and the hollow area is at least one internal cavity of the medical instrument.

In some exemplary embodiments, the contaminated article is contaminated with at least one of a bio-film comprising a plurality of microorganisms, or a plurality of microbial or fungal spores. The contaminated article is exposed to the sterilizing gas containing the acidic and/or oxidizing species for an exposure time sufficient to disinfect the contaminated article by achieving at least a 2-log₁₀ and optionally up to an 11-log₁₀ reduction in colony forming units of the disinfected contaminated article relative to the contaminated article.

In certain such exemplary embodiments, the article undergoing sterilization is a medical device and the enclosed area is a hollow area of the medical device. In some presently-preferred embodiments, the medical device is an endoscope and the hollow area is the lumen of the endoscope, and the sterilizing gas containing the acidic and/or oxidizing species from the plasma generator is passed through the lumen of the endoscope.

In some exemplary embodiments the biofilm comprises a plurality of microorganism species selected from, for example, Geobacillus sp. such as Geobacillus stearothermophilus; Bacillus sp. such as Bacillus subtilis, Bacillus atrophaeus, Bacillus megaterium, Bacillus coagulans, Bacillus pumilus; Clostridium sp. such as Clostridium sporogenes and Clostridium difficile, Aspergillus sp., Aspergillus brasiliensis, Aspergillus oryzae, Aspergillus niger, Aspergillus nidulans, Aspergillus flavus; bacterial cells such as, for example, Mycobacterium terrae, Mycobacterium tuberculosis, and Mycobacterium bovis; and biofilm forming bacteria such as, for example Escherichia coli, Staphylococcus aureus, Pseudomonas aeruginosa, Staphylococcus epidermidis, Staphyolcoccus lugdunensis, Staphylococcus saprophyticus, Staphylococcus epidermidis, Enterococcus faecium, Enterococcus faecalis, Propionobacterium acnes, Klebsiella pneumoniae, Enterobacter cloacae, Proteus mrabilus, Salmonella enterica, Salmonella typhi, Streptococcus mutans, Shigella flexiniri; as well as any combination thereof.

In certain exemplary embodiments, the contaminated article is contaminated with a bio-film including a plurality of microorganisms, the exposure time is at least 5 minutes, and the reduction in colony forming units of the disinfected article relative to the contaminated article is from 4-log₁₀ to 9-log₁₀. More preferably, the reduction in colony forming units of the disinfected article relative to the contaminated article is from 5-log₁₀ to 9-log₁₀; from 6-log₁₀ to 9-log₁₀; or even from 6-log₁₀ to 9-log₁₀.

Preferably, the exposure time to achieve the desired level of disinfection of the contaminated article contaminated with a bio-film including a plurality of microorganisms, is selected to be at most one hour. More preferably, the exposure time to achieve the desired level of disinfection is no greater than 50 minutes, 40 minutes, 30 minutes, 20 minutes, or even 10 minutes. Most preferably, the exposure time to achieve the desired level of disinfection is selected to be at most 9 minutes, 8 minutes, 7 minutes, 6 minutes, 5 minutes, or as low as 4 minutes, 3 minutes, two minutes, or even 1 minute.

In other exemplary embodiments, the contaminated article is contaminated with a bio-film including a plurality of microbial or fungal spores, the exposure time is at least 2 minutes, and the reduction in colony forming units of the disinfected article relative to the contaminated article is from 6-log₁₀ to 10-log₁₀. More preferably, the reduction in colony forming units of the disinfected article relative to the contaminated article is from 7-log₁₀ to 10-log₁₀; from 8-log₁₀ to 10-log₁₀; or even from 9-log₁₀ to 10-log₁₀.

Preferably, the exposure time to achieve the desired level of disinfection of the contaminated article contaminated with a bio-film including a plurality of microbial or fungal spores is selected to be at most one hour. More preferably, the exposure time to achieve the desired level of disinfection is no greater than 50 minutes, 40 minutes, 30 minutes, 20 minutes, or even 10 minutes. Most preferably, the exposure time to achieve the desired level of disinfection is selected to be at most 9 minutes, 8 minutes, 7 minutes, 6 minutes, 5 minutes, or as low as 4 minutes, 3 minutes, two minutes, or even 1 minute.

The operation of exemplary embodiments of the present disclosure will be further described with respect to the following non-limiting detailed Examples. These examples are offered to further illustrate the various specific and preferred embodiments and techniques. It should be understood, however, that many variations and modifications may be made while remaining within the scope of the present disclosure.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Plasma Sterilization Preparatory Examples

The following Preparatory Examples illustrate various plasma sterilization embodiments, which may be practiced in combination with or as modifications to the following Examples, which illustrate embodiments of a combine plasma sterilization and turbulent gas flow drying system and process.

### Materials

Unless otherwise noted, all parts, percentages, ratios, etc. in the Examples and the rest of the specification are by weight. Solvents and other reagents used may be obtained from Sigma-Aldrich Chemical Company (Milwaukee, Wis.) unless otherwise noted. In addition, Table 1 provides abbreviations and a source for all materials used in the Examples below:

TABLE 1
Materials

| Materials | Source |
|---|---|
| Phosphate buffered saline 10X concentrate P5493-1L | Sigma Aldrich, St Louis, MO |
| TWEEN 80 (P4780) | Sigma Aldrich, St Louis, MO |
| 3M ™ Petrifilm ™ Aerobic Count Plates (6400) | 3M Company, St. Paul, MN |
| 3M ™ Petrifilm ™ Plate Reader (6499) | 3M Company, St. Paul, MN |
| Mini Flip Top Vial With Butterfields Buffer | 3M Company, St. Paul, MN |
| Polytetrafluoroethylene (PTFE) tubing, 6.35 mm outer | Sigma-Aldrich Co. LLC, St. Louis, MO |
| diameter/5.80 mm inner diameter (Catalog number 20533) | |
| PTFE Tube 5239K11 | McMaster-Carr, Elmhurst IL |
| PET film 1 mil | Mitsubishi Polyester Film, Greenville SC |
| 3M Color Coded Flat Cable 3302 Series | 3M Company, St. Paul, MN |
| FPG 50-1NM pulse generator | FID GMBH, Burbach, Germany |
| M100B01353CR1BV Mass Flow Controller | MKS Instruments, Andover, MA |
| CT-AQ8G Ozone generator | Ozonefac Co., Guangdong, China |
| 3M Super Rapid Biological Indicator (SRBI) | 3M Company, St. Paul, MN |
| (Nanosilica-primed polypropylene web) | |
| Branson Digital Sonifier | Branson Ultrasonics Co., Danbury, CT |
| Tryptic Soy Agar (TSA) 221283 | Becton Dickinson & Co., Sparks, MD |
| Tryptic Soy Broth (TSB) 297354 | Becton Dickinson & Co., Sparks, MD |
| Geobacillus stearothermophilus (ATCC #7953) | iuvo BioScience LLC, Warsaw, New York |
| Escherichia coli (ATCC ® 25922) | American Type Culture Collection |
| | (ATCC), Manassas, VA |
| Pseudomonas aeruginosa (ATCC ® 15442) | American Type Culture Collection |
| | (ATCC), Manassas, VA |
| Staphylococcus aureus (ATCC ® 6538) | American Type Culture Collection |
| | (ATCC), Manassas, VA |
| Enterococcus faecalis (ATCC ® 14506) | American Type Culture Collection |
| | (ATCC), Manassas, VA |
| Clostridium sporogenes (ATCC ® 3584 ™) | American Type Culture Collection |
| | (ATCC), Manassas, VA |
| Bacillus atrophaeus (ATCC 9372) | iuvo BioScience, Rush, New York |
| Mycobacterium smegmatis (ATCC ® 14468 ™) | American Type Culture Collection |
| | (ATCC), Manassas, VA |
| Sterile 1.5-mL centrifuge tubes | USA Scientific, Ocala, FL |
| Sterile 15-mL conical vials | Corning Mexico, Tamaulipas, Mexico |
| Vortex Mixer | Scientific Industries, Bohemia, NY |
| Sterile tweezers | Thermo Fisher Scientific, Waltham, MA |
| Sterile dissecting scissors | Thermo Fisher Scientific, Waltham, MA |
| Razor blades | Stanley Tools, Towson, MD |
| Isopropyl Alcohol | VWR International, Radnor, PA |

### Procedures

### Preparation of Spore Samples Used in the Examples

First, 1×2 cm films of PET were cut and placed in petri dishes. Then, 10 μL of Geobacillus stearothermophilus spore solution (˜1×10⁸ Colony Forming Units per mL (CFU/mL), vortexed for 1 minute) were drop-cast onto the films. All spores were kept in a refrigerator at 4° C. between uses. The films containing 1×10⁶ spores/film were left to sit with the petri the dish lid open for ≥1 h to ensure that the spore films were fully dry. Next, the films were inserted into 3-inch (7.62 cm) long PTFE sample tubes simulating an endoscope using clean tweezers with 3 films per sample tube. The films were inspected to make sure there was no significant overlap in the spore spot and that the films were in the PTFE tube with the spores face up.

### Collection of Spore Samples and Colony Counting in the Examples

After exposure spore films were removed from PTFE sample tubes using sterile tweezers. Then the films were immediately transferred to 50 milliliter (mL) tubes containing 25 mL of 1× phosphate buffered saline (PBST) to neutralize the pH and all charged plasma species. The 1×PBST was prepared from 100 mL of 10×PBS, 900 mL of deionized water and 1 g of polyethylene glycol sorbitan monooleate surfactant commercially available as TWEEN 80 from Sigma-Aldrich of St. Louis, Mo. The 1×PBST solutions were mixed for 5 minutes on a stir plate and were then vacuum filtered with 0.2 micrometer (m) pore size vacuum filter to ensure sterility and stored at 4° C. The spore films in 1×PBST were vortexed, then sonicated for 20 min and vortexed an additional time to ensure all of the spores were removed from the surface.

One mL of the buffer solution containing spores was diluted in Butterfield's buffer. A serial dilution with 10, 100 and 1000-fold reduction in concentration was carried out because the original samples contained 10⁶ colonies and it was necessary to reduce the concentration enough to count. Then 1 mL of each of the dilution series samples and the original sample in PBST were spread onto disposable spore plates commercially available as PETRIFILM from 3M Company of St. Paul, Minn. The plates were placed onto an aluminum tray and the spores were put in an oven at an optimum growth temperature so that the colonies could grow if CFU were present.

After the spores were incubated, the colony forming units were counted using the PETRIFILM PLATE READER®, commercially available from 3M Company. In each case the control samples of untreated spore films were used as the standard. For ideal quantification of kill, the number of CFU per plate was quantified in the range of 20-200. Based on the number of CFU and the known dilution concentration, it was possible to calculate the number of original CFU from the controls or treated spore films and quantify spore kill.

### Preparative Example 1

A sterilization system generally as described in FIG. 1 and having a plasma generator generally as described in FIG. 2b was provided. More specifically, the plasma generator was constructed by feeding parallel electrodes composed of two strands of 3M Color Coded Flat Cable 3302, commercially available from 3M Company of St. Paul, Minn. into PTFE tubing having a lumen 3/16 inch (4.76 mm) in inside diameter. The anode and cathode were separated on a PVC backing at 0.05 inch (1.27) center-to-center spacing. DC pulsed power was supplied by a power supply commercially available as FPG 50-1NM from FID GmbH of Burbach, DE. The power was set to provide a square pulse with a pulse width of 10 ns and a variable pulse repetition rate and a variable voltage. Power measurements were taken with a homemade E-dot and B-Dot probe.

Flow rates of oxygen and nitrogen gas from tanked sources were controlled using MKS mass flow controllers commercially available from MKS Instruments of Andover, Md. The gasses were mixed and subsequently humidified before being transported to the plasma generator. The plasma byproducts were further transported through connected tubing in order to measure the downstream response. PET film samples which had been inoculated with spores were inserted at recorded lengths within the tubing. In all cases, the spores are downstream from the plasma, outside of the afterglow region. In some cases, the plasma composition was monitored downstream past the spore films using Fourier-Transform Infrared (FTIR) Spectroscopy. An FT-IR spectrometer commercially available as NICOLET iS10 from Thermo Scientific of Waltham, Mass., with a 2-meter gas cell was used to make these measurements. In addition, flow rate was monitored using a flow meter to ensure constant gas/plasma flow between experiments. Standard operating conditions are noted in Table 2. In Preparatory Examples 1-4 below, the standard conditions were used, except where noted. Power was varied by changing both voltage and repetition rate.

TABLE 2
Standard Plasma Sterilization Operating Conditions

| Parameter | Standard Condition | |
|---|---|---|
| Treatment Time | 5 | min |
| Voltage | 33 | kV |
| Flow Rate | 3 | L/min |
| Repetition Rate | 1 | kHz |
| Tube Length | 6 | ft |
| | (about 1.83 m) | |
| Tube ID | 0.1875 | in |
| | (about 4.8 mm) | |
| Humidity | 8.3 | g/m³ |

| | |
|---|---|
| N₂/O₂ Ratio | 80% |

In this example, processing parameters, including voltage, repetition rate (pulse repetition frequency, PRF), and gas flow rate were changed independently and the mean log₁₀ of G. stearothermophilus was observed and recorded. The parameters were altered independently (all others held constant) of values in Table 1. The process values were then normalized according the equation:

$$s / molecule = \frac{\frac{{V}^{2}}{R} \cdot {t}_{ON} \cdot PRF}{pQ / RT}$$

The energy term (V²/R t_{ON}) was taken from I-V measurements on the device. The process values and results are recorded in Table 3.

TABLE 3
Effect of Plasma Power and Gas Flow Rate

| Flow Rate | Power | Exposure Time | Mean Log₁₀ | |
|---|---|---|---|---|
| (Std. L/min) | (watts) | (seconds) | (CFU/Sample) | eV/molecule |
| 0 | 0 | 0 | 6.2 | 0.00 |
| 3 | 12 | 300 | 6.1 | 0.06 |
| 3 | 19 | 300 | 5.2 | 0.09 |
| 3 | 38 | 300 | 0.7 | 0.18 |
| 3 | 45 | 300 | 0.0 | 0.21 |
| 3 | 62 | 300 | 0.0 | 0.29 |
| 3 | 8.06 | 300 | 5.5 | 0.04 |
| 3 | 15.5 | 300 | 5.6 | 0.07 |
| 3 | 31 | 300 | 1.0 | 0.14 |
| 3 | 62 | 300 | 0.0 | 0.29 |
| 0.4 | 62 | 300 | 0.0 | 2.16 |
| 0.5 | 62 | 300 | 0.0 | 1.73 |
| 1 | 62 | 300 | 0.0 | 0.86 |
| 2 | 62 | 300 | 0.5 | 0.43 |
| 3 | 62 | 300 | 0.0 | 0.29 |
| 4 | 62 | 300 | 0.0 | 0.22 |
| 6 | 62 | 300 | 0.0 | 0.14 |
| 8 | 62 | 300 | 4.3 | 0.11 |
| 10 | 62 | 300 | 6.2 | 0.09 |

### Preparative Example 2

The effect of tube volume on kill was investigated by changing the parameter of tube length. All other process parameters were held constant as indicated in Table 1. The results for mean log₁₀ Colony Forming Units (CFU) and the standard deviation (STDEV) about the mean after plasma exposure are recorded in Table 3. A 6-log₁₀ reduction in CFU was observed over the entire range of plasma distance variation from 6 ft. (˜1.85 m) to 300 ft (˜92.52 m). These distances correspond to post plasma residence times ranging from 0.65 second to 32.5 seconds.

TABLE 4
Effect of Distance from Plasma

| Distance | | |
|---|---|---|
| from | | |
| Plasma | | |
| (ft) | Mean Log₁₀ | |
| [m] | CFU/Sample | STDEV |
| Control | 6.07 | 0.24 |
| 6 | 0 | 0 |
| [~1.85] | | |
| 12 | 0 | 0 |
| [~3.71] | | |
| 18 | 0 | 0 |
| [~5.56] | | |
| 24 | 0 | 0 |
| [~7.32] | | |
| 36 | 0 | 0 |
| [~11.10] | | |
| 48 | 0 | 0 |
| [~14.80] | | |
| 75 | 0 | 0 |
| [~23.13] | | |
| 100 | 0 | 0 |
| [~30.84] | | |
| 200 | 0 | 0 |
| [~61.68] | | |
| 300 | 0 | 0 |
| [~92.52] | | |

### Preparative Example 3

In this Preparatory Example, precursors to the plasma were varied independently and the mean log₁₀ CFU and STDEV of G. stearothermophilus was observed and recorded. The conditions of Table 2 were used, except that the ratios of nitrogen to oxygen entering the plasma generator were varied. The results of varying nitrogen and oxygen partial pressures are recorded in Table 5.

TABLE 5
Effect of Varying Nitrogen and Oxygen Partial Pressures

| N₂/N₂ + O₂ | Mean Log₁₀ | |
|---|---|---|
| Ratio | (CFU/Sample) | STDEV |
| Control | 6.2 | 0.3 |
| 0 | 5.9 | 0.3 |
| 0.2 | 0.0 | 0.0 |
| 0.33 | 0.0 | 0.0 |
| 0.5 | 0.0 | 0.0 |
| 0.66 | 0.0 | 0.0 |
| 0.8 | 0.0 | 0.0 |
| 1 | 3.7 | 0.6 |

### Preparative Example 4

In this Preparatory Example, water vapor was added to the gas before directing it to the plasma generator in order to vary the humidity of the sterilizing gas. Otherwise, the conditions of Table 2 were used. The effect of varying the humidity of the sterilizing gas on the mean log₁₀ CFU and STDEV are recorded in Table 6.

TABLE 6
Effect of Varying Sterilizing Gas Humidity

| Humidity | Mean Log₁₀ | |
|---|---|---|
| (g H₂O/m³) | (CFU/Sample) | STDEV |
| Control | 6.4 | 0.1 |
| 3.7 | 5.7 | 0.3 |
| 4.1 | 4.7 | 0.4 |
| 4.5 | 4.9 | 1.0 |
| 5.2 | 0.0 | 0.0 |
| 6.6 | 0.0 | 0.0 |
| 8.5 | 0.0 | 0.0 |
| 9.5 | 0.0 | 0.0 |
| 11.4 | 0.0 | 0.0 |

### Preparative Example 5

A sterilization system generally as described in FIG. 1, and having a plasma generator generally as described in FIG. 2a was provided. More specifically, the ozone generator tube available from Ozonefac Co. (Guangzhou, Guangdong, China) as part of the CT-AQ8G ozone machine was utilized as the plasma electrode. Power was coupled to the plasma electrode from 12 kHz ac power supply with a voltage of 3.6 kV and a total power of 85 W. Oxygen and nitrogen gasses were introduced in the plasma electrode at rates of 0.5 and 2.5 standard liter per minute (SLM), respectively. The gas precursor was humidified with 8.3 g/m³ of vaporized water.

After the plasma generator, the effluent was transported through a 6 foot (˜1.83 m) length of PTFE tubing with a ⅛ inch (˜3.2 mm) diameter inner lumen. The plasma electrode temperature was varied prior to the start of each recording by wrapping heat tape around the plasma electrode system and controlling the temperature to a predetermined setpoint. The mean number of G. stearothermophilus CFU recovered after exposure was observed and recorded. The results are recorded in Table 7.

TABLE 7
Effect of Electrode Temperature

| Electrode | Mean Log₁₀ (CFU/Sample) | |
|---|---|---|
| Temperature | Recovered | |
| (° F.) | 120 second | 300 second |
| [° C.] | Exposure | Exposure |
| Control | 6.3 | |
| 25 | 0.0 | 0.0 |
| [~−3.9° C.} | | |
| 75 | 0.0 | 0.0 |
| [~23.9° C.} | | |
| 100 | 0.0 | 0.0 |
| [~37.8° C.} | | |
| 125 | 0.0 | 0.0 |
| [~51.7° C.} | | |
| 150 | 5.0 | 0.0 |
| [~65.6° C.} | | |
| 175 | 6.3 | 6.0 |
| [~79.4° C.} | | |

### Exemplary Plasma Disinfection Methods for Simulated Endoscope Lumens

The following Preparatory Examples describe a plasma disinfection method useful to achieve microbial kill of mature biofilm found in lumened medical devices such as endoscopes. The plasma disinfection method provides a 6-log reduction in bacteria after 5 minutes of treatment. The disinfection method works on flexible lumened surfaces such as the interior channels of an endoscope. The disinfection method is effective in the presence of moisture and therefore integrates well in the current reprocessing procedures used to decontaminate endoscopes.

If desired, reprocessed endoscopes that have been manually cleaned can be treated with the plasma disinfection method before they are exposed to a high level of disinfection or even sterilization. Alternatively, high level disinfected scopes can be treated with plasma immediately after the automated endoscope reprocessing (AER) cycle and before storage in a drying cabinet. The scopes can also be manually cleaned, disinfected in an AER cycle and stored in a drying cabinet. Plasma treatment can be applied to the stored scopes on demand in a drying cabinet, e.g., prior to patient use to kill any biofilm that may be growing due to improper storage conditions or in the procedure room before use on a patient similar to flash sterilization.

Plasma sterilization or disinfection according to the presently disclosed system and method has also been shown to be effective up to a distance of 6 feet from the source of the plasma, which would accommodate a majority of the endoscopes available on the market today. Plasma disinfection is an on-demand point-of-use disinfection system that is portable and scalable to allow for the treatment of multiple endoscopes at one time.

### Procedures

### Bacterial Culture/Inoculum Preparation

Pseudomonas aeruginosa (ATCC 15442) was subcultured on Tryptic Soy Agar (TSA) plates and incubated at 37° C. for 16 to 18 hours. A single colony was isolated from a streak plate and used to inoculate 10 mL of Tryptic Soy Broth bacterial growth media. A culture was grown at 37° C. for 16 to 18 hours. Viable bacterial density was determined by a ten-fold serial dilution which was plated for enumeration. This was used as the inoculum solution to initiate biofilm growth.

### Biofilm Growth

Four pieces of PTFE tubing 3.28 feet (˜1 length with a 1 mm diameter inner lumen and connector pieces were steam sterilized prior to inoculation. Six hundred microliters of the inoculum were added to fill the entire length of each piece of tubing and biofilm was cultured in each tube for 24 hours at 25° C. The mature biofilm was rinsed with 10% Tryptic. Soy Broth Media to remove planktonic (loosely attached) bacteria for 48 hours. One of the 4 pieces of PTFE tubing was used as a positive control sample to determine the growth of biofilm in the non-disinfected lumens.

### Biofilm Growth Assessment

The positive control PTFE tubing was cut into halves. One half was further cut into four 10 cm sections representing the ends and the middle of the lumen. Each tubing section was placed into a separate sterile Falcon tube containing 15 mL of phosphate buffered saline. The samples were sonicated for 20 minutes at 25° C. The sonicated samples were vortexed and a tenfold serial dilution was made of the PBST used to sonicate each tubing section by transferring 1 mL of the liquid to a sterile conical vial containing 9 mL buffered water.

Dilutions were plated on TSA to determine the population present in the biofilm. The TSA plates were incubated at 23° C.+/−2° C. for a total of 72 hours. The mean population of Pseudomonas aeruginosa (CFU/cm²) present in the mature biofilm was determined and is presented in Table 8.

TABLE 8
Mean population of Pseudomonas aeruginosa
(CFU/cm²) Present in Mature Biofilm

| | | Recovered | | Mean |
|---|---|---|---|---|
| Test | Sample | (CFU) in | Section of the | Population |
| Organism | # | Test Control | Lumen Tested | (CFU/cm²) |
| Pseudomonas | 1 | 2.40E+08 | Front | 2.34E+09 |
| aeruginosa | 2 | 2.46E+08 | Middle #1 | |
| (ATCC 15442) | 3 | 1.85E+08 | Middle #2 | |
| | 4 | 1.19E+08 | End | |

### Preparative Example 6

### Treatment of Polytetrafluoroethylene (PTFE) Tubing Containing Biofilm

A sterilization system generally as described in FIG. 1, and having a plasma generator generally as described in FIG. 2b was provided. More specifically, the plasma generator was constructed by feeding parallel electrodes composed of two strands of 3M Color Coded Flat Cable 3302, commercially available from 3M Company (St. Paul, Minn.) into PTFE tubing having a lumen 3/16 inch (4.76 mm) in inside diameter. The anode and cathode were separated on a PVC backing at a 0.05 inch (1.27 cm) center-to-center spacing.

DC pulsed power was supplied by a power supply commercially available as FPG 50-1NM from FID GmbH (Burbach, Germany). The power was set to provide a rectangular pulse with a pulse width of 10 nanoseconds and a variable pulse repetition rate and a variable voltage. Power measurements were taken with a homemade E-dot and B-dot probe.

The flow rate of compressed air or a nitrogen/oxygen mixture into the PTFE tubing was controlled using an MKS mass flow controller commercially available from MKS Instruments of Andover, Md. The gas was humidified through a bubbling unit before being transported to the plasma generator. The plasma byproducts were further transported through connected tubing in order to measure the downstream response.

The remaining 3 pieces of PTFE tubing inoculated with biofilm were PTFE tubing connected using standard tubing adapters 6 feet (˜1.83 m) downstream from the plasma generator described in Example 1. The operating parameters are recorded below in Table 9.

TABLE 9
Standard Plasma Disinfection Operating Conditions

| Parameter | Standard Condition |
|---|---|
| Treatment Time | 5 min |
| Voltage | 33 kV |
| Flow Rate | 2 L/min |
| Repetition Rate | 1 kHz |
| Tube Length | 6 ft (~1.83 m) |
| Tube ID | 0.1875 in |
| | (~4.8 mm) |
| Gas Temperature | 70° F. (~21.1° C.) |
| Humidity | 100% RH |

The PTFE biofilm tubes contained rinse liquid, which was subsequently blown out of the lumen once the plasma treatment was initiated. This liquid is recorded as the “flow though sample,” and was evaluated by vacuum filtration to determine if any bacteria could be recovered. No bacteria were present in the “flow through sample.”

After plasma treatment, the PTFE tubing was flushed with PBST (1 ml×4) to remove any remaining bacteria which was subsequently recovered by vacuum filtration. Colony forming units counted from this liquid were recorded as “filtrate from wash.” The washed tubing was then cut into sections, placed in a sterile bottle containing 200 mL of PBST and sonicated for 20 mins at 25° C. to remove any biofilm from the lumen of the tubing section. The bacteria present in the sonicated solution was then recovered using vacuum filtration.

The recovered colony forming units after exposure to the plasma are recorded in Table 10. No bacteria were recovered from the “flow through sample,” the “filtrate from wash,” or from the tubing pieces after plasma treatment. Full kill of Pseudomonas aeruginosa present in a mature biofilm (2.34×10⁹ CFU/cm²) was observed after plasma exposure for 5 mins.

TABLE 10
Post Plasma Disinfection Biofilm Recovery

| | | CFU Recovered- |
|---|---|---|
| Sample # | Sample Type | Mean CFU (CFU/cm²) |
| Lumen #1 | Flow through sample | 0 CFU-2.34E+09 CFU/cm² |
| | Filtrate from wash | 0 CFU-2.34E+09 CFU/cm² |
| | Recovery from sonicated | 0 CFU-2.34E+09 CFU/cm² |
| | lumen pieces | |
| Lumen #2 | Flow through sample | 0 CFU-2.34E+09 CFU/cm² |
| | Filtrate from wash | 0 CFU-2.34E+09 CFU/cm² |
| | Recovery from sonicated | 0 CFU-2.34E+09 CFU/cm² |
| | lumen pieces | |
| Lumen #3 | Flow through sample | 0 CFU-2.34E+09 CFU/cm² |
| | Filtrate from wash | 0 CFU-2.34E+09 CFU/cm² |
| | Recovery from sonicated | 0 CFU-2.34E+09 CFU/cm² |
| | lumen pieces | |
| Lumen #4 | Flow through sample | 0 CFU-2.34E+09 CFU/cm² |
| | Filtrate from wash | 0 CFU-2.34E+09 CFU/cm² |
| | Recovery from sonicated | 0 CFU-2.34E+09 CFU/cm² |
| | lumen pieces | |
| Lumen #5 | Flow through sample | 0 CFU-2.34E+09 CFU/cm² |
| | Filtrate from wash | 0 CFU-2.34E+09 CFU/cm² |
| | Recovery from sonicated | 0 CFU-2.34E+09 CFU/cm² |
| | lumen pieces | |
| Lumen #6 | Flow through sample | 0 CFU-2.34E+09 CFU/cm² |
| | Filtrate from wash | 0 CFU-2.34E+09 CFU/cm² |
| | Recovery from sonicated | 0 CFU-2.34E+09 CFU/cm² |
| | lumen pieces | |

### Exemplary Plasma Disinfection Methods for Washed/Undried Lumens

The following Preparatory Example describe a plasma disinfection method useful to achieve microbial kill of biofilm found in washed but undried lumened medical devices such as endoscopes. The Examples show effective kill of four different microorganisms in liquid droplets using two models (10 μL wells and 5.80 mm ID lumens) treated using a remote plasma treatment system and method. These Examples demonstrate disinfection-level kill (≥6 log₁₀) using models that mimic the conditions and residual droplets encountered in the channels of a washed flexible endoscope. This remote plasma system and method is effective at killing microorganisms at a distance of 10 feet (˜3 m) away from the plasma source using an extremely short treatment cycle (e.g., 60-150 seconds).

### Procedures

### Bacterial Culture

Individual streak plates (TSA) of each organism (E. coli, P. aeruginosa, S. aureus, and E. faecalis) were prepared from freezer stocks and incubated for 24 hours at 37° C. A single colony from each plate was used to inoculate 10 mL of TSB growth medium to culture each organism overnight (16-18 hours) with shaking at 250 RPM at 37° C. Each overnight culture reached a concentration ˜10⁹ colony forming units per milliliter (CFU/mL) and was diluted 1:10 in Butterfield's Buffer to create a solution containing ˜10⁸ CFU/mL used to inoculate samples for plasma treatment.

### Plasma Exposure

A plasma disinfection system generally as described in FIG. 1, and having a plasma generator generally as described in FIG. 2a was utilized in examples 7 and 8. Specifically, the ozone generator tube available from Ozonefac Co. (Guangzhou, Guangdong, China) as part of the CT-AQ8G ozone machine was utilized as the plasma electrode. Power was coupled to the plasma electrode from 12 kHz ac power supply with a voltage of 3.6 kV and a total power of 85 W.

Plasma disinfection was achieved by transporting the gas output from the plasma through a 10-foot (˜3 m) length of FEP tubing with an inner diameter of ⅛ inch (˜3.2 mm). Samples were inserted at the end of the 10-foot (˜3 m) tube. The gas output from the remote plasma generator was flowing at a rate of 3 L/min, and the gas was selected to be 1,000 standard cm³/min (SCCM) of moist air and 2000 SCCM of dry air. The relative humidity during all disinfection treatments ranged between 40-60%.

The disinfection treatment cycle for the SRBI wells described in Example 7 consisted of a 150 second plasma exposure followed by a 60 second air flush. Plasma treatments in the Lumen Model (Example 8) ranged from 0-150 seconds followed by a 60 second air flush.

### SRBI Well Sample Preparation

3M SRBI nanosilica primed wells were cut from a roll of the film into individual strips using a standard paper cutter. Each strip contained eight wells capable of holding 10 μL of liquid between the two edges. The strips were cleaned by wiping with 70% isopropyl alcohol and dried prior to use. For each experiment, ˜10⁶ microorganisms were loaded into wells in positions 1 and 8 by pipetting 10 μL of a bacterial suspension containing ˜10⁸ CFU/mL prepared for each organism (E. coli, P. aeruginosa, S. aureus and E. faecalis) as described in the Bacterial Culture section.

For plasma experiments, strips containing the microbial samples were loaded into the 1 foot removable section 6.35 mm outer diameter (OD)/5.80 mm inner diameter (ID) PTFE tubing using sterile tweezers and either treated with a plasma cycle of 150 seconds+60 seconds of air or 210 seconds of air (positive controls). After the plasma exposure, the wells containing the 10 μL samples were cut off from the rest of the strip using sterile dissecting scissors and transferred to individual 1.5-mL tubes containing 1 mL of PBS-TWEEN with sterile tweezers. Each tube was vortex-mixed at maximum speed for 1 minute and serial dilutions were made in Butterfield's Buffer, which were plated on PETRIFILM AEROBIC COUNT plates through the 10⁻⁷ dilution for each sample.

Inoculated plates were incubated for 24-48 hours at 37° C. and counted using a 3M PETRIFILM READER. The 1 minute vortex-mixing in PBS-TWEEN was validated by comparing recovery to enumeration controls (direct serial dilutions of the 10 μL inoculum into Butterfield's Buffer instead of the SRBI well; see Table 11), thereby confirming that this method recovered all of the microorganisms deposited in the SRBI well. For each data point, n=6.

TABLE 11
SRBI Well Sample Recovery Method (1 Minute Vortex) Validation

| | Inoculum | Recovered | Percent |
|---|---|---|---|
| Microorganism | (CFU) | (CFU) | Recovery |
| E coli | 2.68E+06 | 2.63E+06 | 98 |
| P. aeruginosa | 1.63E+06 | 1.60E+06 | 98 |
| S. aureus | 1.83E+06 | 2.30E+06 | 126 |
| E. faecalis | 1.48E+06 | 1.41E+06 | 95 |

### PTFE Lumen Model Sample Preparation

6.35 mm OD/5.80 mm ID PTFE tubing was cut into 6″ lumen sections and steam sterilized prior to use. Samples were inoculated by pipetting 250 μL of a P. aeruginosa culture containing ˜10⁸ CFU/mL into the PTFE lumen. The sample was tilted and rolled to spread the bacteria throughout the tube. The lumens were incubated at room temperature (˜25° C.) for 30 minutes, then most of the liquid (˜150-200 μL) was removed and collected by holding the lumen over a 15-mL conical vial. After this process was completed, visible droplets (˜5-50 μL in volume) still remained in the tube. The inoculated lumens were then attached to the plasma treatment set up as described in the Plasma Exposure section above. Each removable section of tubing was sequentially connected to the 10-foot (˜3 m) piece. A time-course experiment was conducted with duplicate samples exposed to a plasma disinfection treatment cycle of 0 seconds, 15 seconds, 30 seconds, 60 seconds, 90 seconds, 120 seconds and 150 seconds, with each plasma exposure followed by a 60 second air flush.

After each disinfection treatment cycle was complete, each lumen was cut in half to create two 3-inch (˜7.62 cm) sections using a razor blade freshly cleaned with isopropyl alcohol and transferred to a 15-mL conical vial containing 10 mL of PBS-TWEEN. Any remaining viable bacteria were then recovered from each lumen by vortex-mixing the vial at maximum speed for 1 minute, disrupting with 2×20 second duration pulses at 20 kHz with a probe sonicator set at 39% of the maximum amplitude, then vortex-mixing again for 1 minute at maximum speed.

Serial dilutions of each tube were made in Butterfield's Buffer and plated on PETRIFILM® AEROBIC COUNT plates through the 10⁻⁷ dilution for each sample (with the original 10 mL recovery solution being the 10¹ dilution). The inoculated plates were incubated for 24-48 hours at 37° C. and counted using a 3M PETRIFILM READER. This lumen test procedure was adapted from the American Society for Testing and Materials International (ASTM) method E1837—Standard Test Method to Determine Efficacy of Disinfection Processes for Reusable Medical Devices (Simulated Use Test).

### Preparative Example 7

Bacterial Kill from 150 sec Plasma Treatment in Liquid Droplets in 10 μL Wells.

Six replicate samples of each microorganism were exposed to a 150 second duration plasma treatment and a 60 second duration air purge at a flow rate of 3 L/min at a distance to 10 feet (˜3 m) away from the plasma source. Each sample contained ˜10⁶ viable cells in 10 μL of liquid when the plasma treatment began.

Measuring the mass of the SRBI strips before and after plasma the treatment cycle revealed that a mean of 0.0011 g (˜1.1 μL) of the 10 μL droplet (n=24) evaporated during the plasma exposure (data not shown). After the plasma cycle, no viable colony forming units were recovered from the samples exposed to plasma, and full kill (6+ logs; Table 12) was achieved for all four microorganisms tested (E. coli, P. aeruginosa, S. aureus and E. faecalis). These four organisms were chosen as representative examples of both Gram-negative and Gram-positive bacteria and due to their relevance as organisms of “high concern” for endoscope reprocessing as stated by the Centers for Disease Control and Prevention (CDC). (see, e.g., “Interim Protocol for Healthcare Facilities Regarding Surveillance for Bacterial Contamination of Duodenoscopes after Reprocessing” which can be found on the CDC website (https://www.cdc.gov/hai/pdfs/cre/interim-duodenoscope-surveillance-Protocol.pdf, published Mar. 11, 2015, last accessed Jun. 12, 2017). For each data point, the number of samples n=6.

TABLE 12
Bacterial Kill from 150 second Plasma Treatment in 10 μL Wells

| | No Plasma | 150 seconds Plasma | |
|---|---|---|---|
| | Positive Control | Treatment | |
| | (Mean Recovered | (Mean Recovered | Log₁₀ |
| Microorganism | CFU) | CFU) | Reduction |
| E. coli | 6.76E+06 | 0.00E+00 | 6.83 |
| P. aeruginosa | 1.48E+06 | 0.00E+00 | 6.17 |
| S. aureus | 5.89E+06 | 0.00E+00 | 6.77 |
| E. faecalis | 1.29E+06 | 0.00E+00 | 6.11 |

### Preparative Example 8

### Bacterial Kill Curve in Liquid Droplets in PTFE Lumens

A kill curve in a 5.80 mm ID PTFE lumen model based on ASTM E1837 was generated by exposing duplicate samples incubated with P. aeruginosa suspensions to plasma cycles of varying exposure time from 0 second-150 seconds. Each lumen contained droplets ranging from ˜5-50 μL when plasma treatment was initiated. Visible droplets remained in the lumen after plasma exposure, but the amount of residual liquid was not quantified. Recovery of any remaining viable bacteria after the plasma cycle showed the complete kill (7.6-log₁₀) was achieved within 60 seconds of plasma treatment (Table 13).

TABLE 13
Survival of P. aeruginosa in a 5.80 mm ID Lumen over Time.

| Exposure | Mean Recovered |
|---|---|
| time | CFU/sample |
| (sec) | (n = 2) |
| 0 | 3.87E+07 |
| 15 | 2.85E+05 |
| 30 | 2.10E+02 |
| 60 | 0.00E+00 |
| 90 | 0.00E+00 |
| 120 | 0.00E+00 |
| 150 | 0.00E+00 |

### Exemplary Plasma Sterilization and Turbulent Flow Drying Methods

The following Examples illustrate the combination of plasma sterilization alternating with with turbulent flow drying using a drying gas. It will be understood that any of the plasma sterilization embodiments illustrated in the Preparatory Examples may be combined with the turbulent flow drying embodiments in the following illustrative Examples.

Unless otherwise noted, all parts, percentages, ratios, etc. in the Examples and the rest of the specification are by weight. Solvents and other reagents used may be obtained from Sigma-Aldrich Chemical Company (Milwaukee, Wis.) unless otherwise noted. In addition, Table 14 provides abbreviations and a source for all materials used in the Examples below:

TABLE 14
Materials

| Material | Source |
|---|---|
| MF0335 Metric PTFE Tubing, | Cole Parmer (Vernon Hills, IL) |
| 2.48 mm × 4 mm (Item EW-06605-53) | |
| Autoclave Indicator Tape | Fisher Scientific (Waltham, MA) |
| Pseudomonas aeruginosa (ATCC ® | American Type Culture Collection (Manassas, VA) |
| 15442) | |
| Tryptic Soy Agar (TSA) | Becton, Dickinson and Company (Sparks, MD) |
| Tryptic Soy Broth (TSB) | Becton, Dickinson and Company (Sparks, MD) |
| Polysorbate 80 | Alfa Aesar (Ward Hill, MA) |
| Lecithin | Alfa Aesar (Ward Hill, MA) |
| L-histidine | Alfa Aesar (Ward Hill, MA) |
| Sodium Thiosulphate | Alfa Aesar (Ward Hill, MA) |
| Molecular Grade Demineralized Water | Fisher Scientific (Waltham, MA) |
| Tryptone | Becton, Dickinson and Company (Sparks, MD) |
| Sodium Chloride | Fisher Scientific (Waltham, MA) |
| 70% Isopropyl Alcohol | VWR Chemicals (Radnor, PA) |
| Breathable Peel Open Pouch for | 3M Company (Saint Paul, MN) |
| Ethylene Oxide and 3M OPTREOZ 125- | |
| Z Sterilizers | |
| 9-mL Mini Flip Tops of Butterfield's | 3M Company (Saint Paul, MN) |
| Buffer | |
| PetriFilm ® Aerobic Count Plates | 3M Company (Saint Paul, MN) |
| Wassenburg Endoscope Surrogate | Wassenberg Medical B.V. (Dodewaard, the |
| Device | Netherlands) |

### Equipment

The following laboratory equipment was used in carrying out the Examples:

Branson Digital Sonifier, Branson Ultrasonics (Danbury, Conn.)

Impulse Tabletop Poly Bag Heat Sealer, Uline, Corp. (Minneapolis, Minn.)

Vortex Mixer, Scientific Industries, Inc. (Bohemia, N.Y.)

### Procedures

### Preparation of EN16442:2015 Annex E Media:

The Sampling Solution and Diluent were prepared as described in the BS EN16442:2015 Annex E Standard in sections E.1.3.3 and E1.3.4, respectively. The Sampling Solution contained 3 mL polysorbate 80, 0.3 g lecithin, 0.1 g L-histidine, and 0.5 g sodium thiosulphate and was diluted to a total volume of 100 mL with demineralized water. The Diluent Solution contained 26.22 g tryptone and 7.78 g sodium chloride prepared in 1 L of demineralized water. Both solutions were steam sterilized using a 20-minute cycle time prior to use.

### Bacterial Culture

A streak plate of Pseudomonas aeruginosa (ATCC 15442) was prepared from a frozen stock on Tryptic Soy Agar and left at 37° C. overnight to incubate. A single colony from the plate was used to inoculate 10 mL of sterile Tryptic Soy Broth and grown overnight with shaking at 250 RPM at 37° C. The overnight culture (approximately 10⁹ colony forming units (CFU)/mL) was diluted 1:10,000 in BS EN16442:2015 Annex E Diluent solution. This dilution was used to inoculate all samples.

### Sample Preparation (See BS EN16442:2015 Annex E E.1.4.5)

A 2.48 mm inner diameter PTFE tubing was cut into 1.25 m sections and a 1/16″ (about 1.6 mm) female Luer connector was inserted into one end of each tubing section. The pieces of tubing were coiled and individually wrapped in aluminum foil then steam sterilized using a 20-minute cycle. Each piece of tubing was prepared as described in BS EN16442:2015 Annex E section.

Samples were contaminated by drawing 4 mL of the P. aeruginosa-inoculated EN16442 diluent into a 5-mL syringe, locking the syringe into the female Luer connector attached to one end of the tubing, and transferring the liquid from the syringe to the PTFE tubing. Each contaminated sample was incubated at room temperature for 60 minutes. The bulk of the contaminated liquid was removed by purging the tubing with 50 mL of air using a 60-mL syringe attached through the Luer connector. The female Luer connector was removed and the exterior of each sample was cleaned by wiping it with 70% isopropyl alcohol.

### Sample Treatment and Drying

Each contaminated tubing sample was connected to the suction/biopsy channel of a Wassenburg Endoscope Surrogate Device (Wassenberg Medical B.V., Dodewaard, the Netherlands) for plasma treatment individually. A remote plasma treatment system as described above was used. The plasma treatment and drying cycle consisted of a 10 second (sec) air purge at 25 psig (about 172,369 Pa), 90 sec of plasma flowing at 3 L/minute, and then 260 sec of heated air (60° C.) at 25 psig (about 172,369 Pa).

During the plasma cycle, power was coupled to the plasma electrode from 12 kHz ac power supply with a voltage of 3.6 kV and a total power of 85 W. During the plasma cycle, humidified air was introduced in the plasma electrode at a rate of 3 standard liter per minute (slm). The air was humidified with 40% RH at 21° C.

After the treatment and drying cycle, each sample was disconnected from the Wassenburg Device and the exterior was cleaned by wiping with 70% isopropyl alcohol. Each tube was coiled and placed in a 3M Breathable Peel Open Pouch for storage. Each pouch was sealed closed using a tabletop heat sealer.

The plasma treated samples and controls were stored at room temperature (20-25° C.) for periods of 0, 24, 48, 168, and 720 hours. Duplicate samples, untreated positive controls, and negative controls were all done in duplicate, per the BS EN16442:2015 Annex E standard.

### Sample Recovery

Any remaining viable P. aeruginosa were recovered from each sample as follows. A 20 mL aliquot of EN16442:2015 Annex E Sampling Solution was transferred to a sterile 50-mL conical vial. The tubing sample was removed from the sealed storage pouch and a sterile female Luer connected was inserted into one end of the tubing. The exterior of the sample was cleaned by wiping with 70% isopropyl alcohol and the end of the tubing without the Luer connector was inserted into the bottom of the conical vial containing the 20 mL aliquot of Sampling Solution.

A 20-mL Luer lock syringe was attached to the opposite end of the tubing via the female Luer connecter. The 20 mL aliquot of Sampling Solution was washed through the lumen of the tubing by drawing the liquid into the syringe and pushing it back into the 50-mL conical vial. This was repeated a total of five times.

The 20-mL syringe was then removed and a 60-mL syringe was used to purge remaining liquid from the lumen by forcing through 50 mL of air twice (a total of 100 mL) into the conical vial containing the sample. The vial was capped and then vortexed at maximum speed for 1 minute.

Serial dilutions of the recovered sample were made in Butterfield's Buffer and plated on PetriFilm Aerobic Count plates through the −10 dilution (with the original 20-mL aliquot of sampling solution serving as the −1 dilution). Plates were incubated for 24-48 hours at 37° C. and counted using a 3M PetriFilm Reader.

### Validation of Sampling Method (See BSEN16442:2015 Annex E, E.1.4.7.2):

As described in EN16442:2015 Annex E, storage times of longer than 12 hours can lead to fixation of bacteria and biofilm formation. This may make it difficult to remove bacteria from the inner lumen of the tubing samples. The following procedure was performed after the Sample Recovery method described above to validate that the method adequately removed the bacteria.

After recovery was performed, each sample was brushed using the small end of an Olympus Single Use Combination Cleaning Brush per the manufacturer's instructions. The brush was dipped in a new 20 mL aliquot of Sampling Solution, then forced back and forth through the lumen of the tubing sample a total of three times while actively brushing. The head of the brush was then cut off and submerged in the 20 mL of sampling solution.

The vial was capped, vortexed at maximum speed for 1 minute, bacteria were dislodged from the brush head with 2×20 sec pulses at 20 kHz using a probe sonicator set at 39% of the maximum amplitude, then vortexed for 1 minute at maximum speed a second time.

Serial dilutions of the recovered sample were made in Butterfield's Buffer and plated on PetriFilm® Aerobic Count plates through the −10 dilution (with the original 20-mL aliquot of sampling solution serving as the −1 dilution). Plates were incubated for 24-48 hours at 37° C. and counted using a 3M PetriFilm® Reader.

Additionally, the same Sample Recovery procedure and plating performed above was repeated after brushing. The acceptance criteria for validation of the recovery method states that the number of CFU recovered from the brush and post brushing steps must be less than the first recovery step. The CFU counts from the brush and the post brushing step remained 1-2 orders of magnitude less than the first recovery step for all storage times (0, 24, 48, 168 and 720 h), which met the acceptance criteria in the standard.

### Example 1

### Elimination of Residual Moisture in Lumen Channels Using High Pressure Air

Current industry standards provide minimal direction as to how to adequately dry the endoscope channels. In order to meet the criteria defined in BS EN16442:2015, after the indicated treatment there cannot be any observed droplets removed from the lumen channels with a minimal air flush (up to 17 psig, about 117,211 Pa). Therefore, even being able to meet this criterion could still mean residual droplets are left within the endoscope channels that could contribute to microbial growth over time.

Samples of 2.48 mm ID PTFE tubing (1.25 meter length) were dosed with 10 mL of sterile water. Each sample was flushed with air at a specified air pressure of 10 psig (about 68,948 Pa) or 17 psig (about 117,211 Pa) after the 90 sec plasma treatment. During the air flush, the tubing was qualitatively evaluated to determine the exposure time at which evaporation of the last remaining water droplet occurred. This Example demonstrates the ability to meet the BS EN16442:2015 criteria rapidly within 20 seconds at 17 psig (about 117,211 Pa), as shown in Tables 15 and 16. This Example also demonstrates the ability to completely remove any residual droplets after longer flush times, as shown in Tables 15 and 16.

TABLE 15
Drying Time Required to Meet EN16442:2015 Standard or Remove
All Visible Droplets in Lumen (10 psig (about 68,948 Pa) gas pressure)

| Drying Time After | Meets | |
|---|---|---|
| 90 sec Plasma Treatment | EN16442:2015 | Removal of All Visible |
| (Minutes:Seconds) | Standard? | Droplets in Lumen? |
| 00:05 | No | No |
| 00:10 | No | No |
| 00:15 | No | No |
| 00:20 | No | No |
| 00:25 | No | No |
| 00:30 | Yes | No |
| 03:11 | Yes | Yes |

TABLE 16
Drying Time Required to Meet EN16442:2015 Standard or Remove
All Visible Droplets in Lumen (17 psig (about 117,211 Pa) gas pressure)

| Dry Time-After | Meets | Removal of All |
|---|---|---|
| 90 sec Plasma | EN16442:2015 | Visible Droplets |
| Treatment (MM:SS) | Standard | in Lumen |
| 00:05 | No | No |
| 00:10 | No | No |
| 00:15 | No | No |
| 00:20 | Yes | No |
| 02:09 | Yes | Yes |

### Example 2

Microbial Kill and Growth Prevention after Plasma Sterilization and Drying

### (10 sec High Pressure Air, 90 sec Plasma Treatment, and 260 sec High Pressure Hot Air Drying)

Using a Wassenburg Endoscope Surrogate Device and protocol based on BS EN16442:2015, duplicate samples incubated with P. aeruginosa suspensions were exposed to plasma cycles consisting of a 10 sec, 25 psig (about 172,368 Pa) air purge, 90 sec plasma treatment flowing at 3 L/minute, and then 260 sec, 25 psig (about 172,368 Pa) heated (60° C.) air purge, as described above in Sample Treatment and Drying. Recovery of any remaining viable bacteria after the plasma cycle showed that complete kill (7.6 log₁₀) was achieved and maintained with plasma treatment over the course of the 720-hour storage condition (Table 17).

TABLE 17
Average P. aeruginosa CFU recovered from treated (according to
Example 2) and untreated 2.48 mm ID lumen in a Wassenburg
Endoscope Surrogate Device

| | Average CFU Recovered (n = | |
|---|---|---|
| Storage Time | 2) | |
| (hours) | Treated | Control |
| 0 | 0.0 | 4.6 × 10⁶ |
| 24 | 0.0 | 3.8 × 10⁹ |
| 48 | 0.0 | 7.9 × 10⁹ |
| 168 | 0.0 | 5.3 × 10⁹ |
| 720 | 0.0 | 1.4 × 10⁷ |

Additionally, separate samples incubated with P. aeruginosa suspensions were exposed to the plasma cycle, an air only cycle, or remained untreated and were evaluated over a 48 hour period. Recovery of any remaining viable bacteria showed that the plasma treatment is critical to achieving complete kill of the microorganism. Air drying alone is not sufficient to demonstrate full kill as shown in Table 18.

TABLE 18
Average P. aeruginosa log change from plasma treated versus air
drying only and untreated 2.48 mm ID lumen in Wassenburg
Endoscope Surrogate Device.

| | Average CFU Recovered and [log change] | | |
|---|---|---|---|
| | (n = 2) | | |
| Storage Time | Plasma | Air Purge | Untreated |
| (hours) | Treated | Only | Control |
| 0 | 0.00 | 5.68 × 10³ | 1.90 × 10⁹ |
| | [−6.66 log] | [−2.91 log] | [+2.62 log] |
| 48 | 0.00 | 3.50 × 10² | 3.95 × 10⁹ |
| | [−6.66 log] | [−4.12 log] | [+2.94 log] |

Reference throughout this specification to “one embodiment,” “certain embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus the appearances of the phrases such as “in one or more embodiments,” “in certain embodiments,” “in one embodiment” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

Additionally, all publications and patents referenced herein are incorporated by reference in their entirety to the same extent as if each individual publication or patent was specifically and individually indicated to be incorporated by reference. Various exemplary embodiments have been described.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. These and other embodiments are within the scope of the following claims.

1. A system for sterilizing a contaminated article, comprising:
a source of a drying gas configured to provide a turbulent flow of the drying gas to dry the contaminated article;
a plasma generator having:
an electrode,
a shield, and
a dielectric gap between the electrode and the shield;
a source of electrical power connected to the plasma generator for applying an electrode energy density between the electrode and the shield; and
a source of a sterilizing gas precursor comprising water vapor, oxygen, and nitrogen, configured to provide a flow of the sterilizing gas precursor through the plasma generator between the electrode and the shield to form a plasma, wherein a temperature at the surface of the shield is maintained at less than 150° C. when the electrode energy density is greater than 0.05 eV/molecule of the sterilizing gas precursor passing between the electrode and the shield, further wherein the plasma forms from the sterilizing gas precursor a sterilizing gas comprising acidic and/or oxidizing species, and further wherein the contaminated article is exposed to a flow of the sterilizing gas, optionally wherein the system further comprises a device for conveying the contaminated article through a chamber fluently connected to the flow of the sterilizing gas.

2. The system of claim 1, wherein the sterilizing gas includes one or more species selected from the group consisting of molecular oxygen, molecular nitrogen, nitric oxide, nitric acid, and nitrous oxide.

3. The system of claim 2, wherein the sterilizing gas precursor comprises air, optionally wherein a relative humidity of the sterilizing gas precursor is at least 21%.

4. The system of claim 1, further comprising one or more valves configured to alternate the flow of the drying gas and the flow of the sterilizing gas to the contaminated article, a cooling apparatus, a filter for removing the acidic and/or oxidizing species from the sterilizing gas, or a combination thereof.

5. (canceled)

6. The system of claim 1, wherein the source of electrical power is a pulsed DC source having a high dV/dT.

7. (canceled)

8. A method of sterilizing a contaminated article, comprising:
providing a sterilizer including:
a source of a drying gas configured to provide a turbulent flow of the drying gas to dry the contaminated article;
a plasma generator including:
an electrode,
a shield, and
a dielectric gap between the electrode and the shield;
a source of electrical power connected to the plasma generator for applying an electrode energy density between the electrode and the shield; and
a source of a sterilizing gas precursor comprising water vapor, oxygen, and nitrogen, configured to provide a flow of the sterilizing gas precursor through the plasma generator between the electrode and the shield to form a plasma containing acidic and/or oxidizing species from the sterilizing gas precursor;
providing the flow of the sterilizing gas precursor through the plasma generator between the electrode and the shield to form the plasma, wherein a temperature at the surface of the shield is maintained at less than 150° C. when the electrode energy density is greater than 0.05 eV/molecule of the sterilizing gas precursor passing between the electrode and the shield, further wherein the plasma causes the flow of sterilizing gas precursor to form a flow of a sterilizing gas comprising the acidic and/or oxidizing species;
directing the flow of the sterilizing gas containing the acidic and/or oxidizing species from the plasma generator through an enclosed space enclosing at least a portion of the contaminated article;
exposing the contaminated article to the sterilizing gas containing the acidic and/or oxidizing species for an exposure time sufficient to achieve a desired degree of sterilization of the contaminated article, optionally wherein the time sufficient to achieve the desired degree of sterilization of the contaminated article is no greater than one hour; and
directing a turbulent flow of the drying gas into the enclosed space to dry the contaminated article,
optionally further comprising removing at least a portion of the acidic and/or oxidizing species from the sterilizing gas after the sterilizing gas is directed through the enclosed space.

9. (canceled)

10. The method of claim 89, wherein the removing at least a portion of the acidic and/or oxidizing species from the sterilizing gas is performed with a filter comprising one or more materials selected from the group consisting of activated carbon, a species with a basic functionality, a species providing a basic adsorbent, a reducing species, and a molecular sieve.

11. The method of claim 8, wherein the enclosed space is a sterilization chamber into which the contaminated article is placed.

12. The method of claim 8, wherein the directing the flow of the sterilizing gas through the enclosed space occurs for a duration of at least 10 sec and no more than 5 min, and is followed by the directing the flow of the drying gas through the enclosed space for a duration of at least 10 sec and no more than 10 min, optionally wherein the directing the flow of the sterilizing gas and the directing the flow of the drying gas are alternately repeated at least twice.

13. The method of claim 8, wherein at least one of the drying gas, the sterilizing gas precursor, or the sterilizing gas has a temperature of from 10° C. to 60° C.

14. The method of claim 8, wherein the drying gas is selected from the group consisting of oxygen, nitrogen, helium, neon, argon, krypton, or a combination thereof, optionally wherein the drying gas is substantially free of water.

15. The method of claim 8, wherein the sterilizing gas includes one or species selected from the group consisting of molecular oxygen, molecular nitrogen, nitric oxide, nitric acid, and nitrous oxide.

16. The method of claim 8, wherein the sterilizing gas precursor comprises air, optionally wherein a relative humidity of the sterilizing gas precursor entering the plasma generator is at least 21%.

17. The method of claim 8, wherein the source of electrical power is a pulsed DC source having a high dV/dT.

18. The method of claim 8, wherein the contaminated article is a medical device and the enclosed space is a hollow area of the medical device.

19. The method of claim 18, wherein the medical device is an endoscope and the hollow area is a lumen of the endoscope, further wherein the sterilizing gas containing the acidic and/or oxidizing species from the plasma generator is passed through the lumen of the endoscope.

20. The method of claim 18, wherein the medical device is a medical instrument and the hollow area is at least one internal cavity of the medical instrument.

21. The method of claim 8, wherein the contaminated article is contaminated with at least one of a bio-film comprised of a plurality of microorganisms, a plurality of microorganisms, a bio-film comprised of a plurality of microbial spores, a plurality of microbial spores, a bio-film comprised of a plurality of fungi, or a plurality of fungi, optionally wherein the bio-film comprises a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Streptococcus mutans, Shigella flexiniri, and combinations thereof.

22. (canceled)

23. The method of claim 21, wherein the contaminated article is contaminated with a bio-film comprising a plurality of microorganisms, further wherein the exposure time is at least 5 minutes, and the reduction in colony forming units of the disinfected article relative to the contaminated article is from 4-log₁₀ to 9-log₁₀, optionally wherein the exposure time is at most one hour.

24. The method of claim 21, wherein the contaminated article is contaminated with a bio-film comprising a plurality of microbial or fungal spores, further wherein the exposure time is at least 2 minutes, and the reduction in colony forming units of the disinfected article relative to the contaminated article is from 6-log₁₀ to 10-log₁₀, optionally wherein the exposure time is at most one hour.
